SEQUENCE LISTING (30 sequences)

SEQ ID NO: 1 - protein, 22 aa, foot and mouth disease virus (FMDV)
VKQTLNFDLLKLAGDVESNPGP

SEQ ID NO: 2 - protein, 18 aa, Thosea asigna virus
EGRGSLLTCGDVEENPGP

SEQ ID NO: 3 - protein, 19 aa, porcine teschovirus-1
ATNFSLLKQAGDVEENPGP

SEQ ID NO: 4 - protein, 20 aa, equine rhinitis A virus (ERAV)
QCTNYALLKLAGDVESNPGP

SEQ ID NO: 5 - protein, 22 aa, Bombyx mori cytoplasmic polyhedrosis virus (BmCPV)
DVFRSNYDLLKLCGDIESNPGP

SEQ ID NO: 6 - protein, 22 aa, Bombyx mori infectious flacherie virus (BmIFV)
TLTRAKIEDELIRAGIESNPGP

SEQ ID NO: 7 - protein, 8 aa, Artificial Sequence
  Inducing ribosome skipping during eukaryotic protein translation
  VARIANT (2)..(2): Val or Ile
  misc_feature (4)..(4): Xaa can be any naturally occurring amino acid
DVEXNPGP

SEQ ID NO: 8 - protein, 147 aa, Saccharomyces cerevisiae
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 9 - protein, 95 aa, Saccharomyces cerevisiae
MSSDSSKIKRKRNRIPLSCTICRKRKVKCDKLRPHCQQCTKTGVAHLCHYMEQTWAEEAEKELLKDNELKKLRERVKSLEKTLSKVHSSPSSNST

SEQ ID NO: 10 - protein, 201 aa, Escherichia coli
KALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWL

SEQ ID NO: 11 - protein, 78 aa, herpes simplex virus
APPTDVSLGDELHLDGEDVAMAHADALDDFDLDMLGDGDSPGPGFTPHDSAPYGALDMADFEFEQMFTDALGIDEYGG

SEQ ID NO: 12 - protein, 71 aa, Zea mays
MAGGGGGGGGEAGSSDDCSSAASVSLRVGSHDEPCFSGDGDGDWMDDVRALASFLESDEDWLRCQTAGQLA

SEQ ID NO: 13 - DNA, 95 bp, Artificial Sequence
  Gal4-binding sequence
cggagtactgtcctccgagcggagtactgtcctccgagcggagtactgtcctccgagcggagtactgtcctccgagcggagtactgtcctccgag

SEQ ID NO: 14 - DNA, 100 bp, Artificial Sequence
  Hap1-binding sequence
agcacggacttatcggtcggagcacggacttatcggtcggagcacggacttatcggtcggagcacggacttatcggtcggagcacggacttatcggtcgg

SEQ ID NO: 15 - DNA, 100 bp, Artificial Sequence
  LexA-binding sequence
tactgtatatatatacagtatactgtatatatatacagtatactgtatatatatacagtatactgtatatatatacagtatactgtatatatatacagta

SEQ ID NO: 16 - DNA, 47 bp, cauliflower mosaic virus
gcaagacccttcctctatataaggaagttcatttcatttggagagga

SEQ ID NO: 17 - DNA, 239 bp, Drosophila melanogaster
ccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctg

SEQ ID NO: 18 - DNA, 95 bp, Cytomegalovirus (CMV)
ccaagcagagctcgtttagtgaaccgtcagatcgcctggagacgccatccacgctgttttgacctccatagaagacaccgggaccgatccagcct

SEQ ID NO: 19 - DNA, 1167 bp, Artificial Sequence
  self-amplifying expression system for insulin production
  repeat_region (1)..(95): 5X UAS
  TATA_signal (96)..(142): minimal cauliflower mosaic virus 35S promoter (CaMV 35S)
  CDS (143)..(661): Hap1VP16 transcription factor
  CDS (662)..(715): T2A peptide
  CDS (716)..(979): mature human insulin coding region
  terminator (980)..(1167): yeast ADH1 terminator
cggagtactgtcctccgagcggagtactgtcctccgagcggagtactgtcctccgagcggagtactgtcctccgagcggagtactgtcctccgaggcaagacccttcctctatataaggaagttcatttcatttggagaggaatgtcctccgactcgtccaagatcaagaggaagcggaaccgcatcccgctcagctgcaccatctgccggaagaggaaggtcaagtgcgacaagctcaggccgcactgccagcagtgcaccaagaccggggtggcccacctctgccactacatggagcagacctgggccgaggaggccgagaaggagttgctgaaggacaacgagttgaagaagctcagggagcgcgtgaagtccttggagaagaccctctccaaggtgcactcctccccgtcgtccaactccacggcccccccgaccgacgtcagcctgggggacgagctccacttagacggcgaggacgtggcgatggcgcatgccgacgcgctagacgatttcgatctggacatgttgggggacggggattccccggggccgggatttaccccccacgactccgccccctacggcgctctggatacggccgacttcgagtttgagcagatgtttaccgatgcccttggaattgacgagtacggtggggaaggaagggggtcactattgacgtgtggtgatgtggaggagaacccaggcccaatgttcgttaaccagcacctgtgtggctctcacctcgtggaggctctgtatctggtctgcggagaaagaggcttcttctacactcctaagacccggagggaagcagaggatcttcaagttggacaggtggagctcggtggaggcccaggcgcgggttccctgcaaccgttggcgcttgagggatccttgcaaaagaggggaattgtagagcaatgctgcacctccatctgttcattgtatcagctcgaaaactattgcaattgagcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccgg

SEQ ID NO: 20 - protein, 173 aa, Artificial Sequence
  Synthetic Construct
MSSDSSKIKRKRNRIPLSCTICRKRKVKCDKLRPHCQQCTKTGVAHLCHYMEQTWAEEAEKELLKDNELKKLRERVKSLEKTLSKVHSSPSSNSTAPPTDVSLGDELHLDGEDVAMAHADALDDFDLDMLGDGDSPGPGFTPHDSAPYGALDTADFEFEQMFTDALGIDEYGG

SEQ ID NO: 21 - protein, 18 aa, Artificial Sequence
  Synthetic Construct
EGRGSLLTCGDVEENPGP

SEQ ID NO: 22 - protein, 87 aa, Artificial Sequence
  Synthetic Construct
MFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 23 - DNA, 1464 bp, Artificial Sequence
  A self-amplifying preproinsulin gene expression system in a Lentiviral vector
  repeat_region (1)..(95): 5X UAS
  TATA_signal (96)..(299): basal cytomegalovirus (CMV) promoter
  sig_peptide (367)..(438): insulin signal peptide
  CDS (367)..(696): preproinsulin
  CDS (697)..(762): F2A peptide
  CDS (763)..(1464): Gal4VP16 transcription factor (TF)
cggagtactgtcctccgagcggagtactgtcctccgagcggagtactgtcctccgagcggagtactgtcctccgagcggagtactgtcctccgaggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaacgtgaaacagactttgaattttgaccttctcaagttggcaggagacgtggagtccaacccagggcccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggaattcccggggatctggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagtaatga

SEQ ID NO: 24 - protein, 110 aa, Artificial Sequence
  Synthetic Construct
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 25 - protein, 22 aa, Artificial Sequence
  Synthetic Construct
VKQTLNFDLLKLAGDVESNPGP

SEQ ID NO: 26 - protein, 231 aa, Artificial Sequence
  Synthetic Construct
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGG

SEQ ID NO: 27 - DNA, 4522 bp, Artificial Sequence
  Fib-H-piggybac-Hap1VP1
  promoter (1)..(1076): Fib-H endogenous promoter (partial genomic DNA)
  repeat_region (1077)..(1311): piggybac 3′ inverted terminal repeat sequences (ITR)
  promoter (1312)..(1543): 3X P3 promoter
  CDS (1544)..(2221): dsRed reporter protein
  polyA_signal (2342)..(2463): SV40 poly(A) signal
  repeat_region (2462)..(2776): piggybac 5′  inverted terminal repeat sequences (ITR)
  repeat_region (2777)..(2871): 5X UAS
  TATA_signal (2872)..(2918): minimal cauliflower mosaic virus promoter (CaMV 35S)
  CDS (2919)..(3437): Hap1VP16 transcription factor (TF)
  CDS (3438)..(3491): T2A peptide
  gene (3492)..(4522): Fib-H endogenous coding region (partial genomic DNA)
tacttatcacaacttgtttttataataattcgcttaaatgagcagctattacttaatctcgtagtggtttttgacaaaatcagcttctttagaactaaaatatcatttttttcgtaatttttttaatgaaaaatgctctagtgttatacctttccaaaatcaccattaattaggtagtgtttaagcttgttgtacaaaactgccacacgcatttttttctccactgtaggttgtagttacgcgaaaacaaaatcgttctgtgaaaattcaaacaaaaatattttttcgtaaaaacacttatcaatgagtaaagtaacaattcatgaataatttcatgtaaaaaaaaaatactagaaaaggaatttttcattacgagatgcttaaaaatctgtttcaaggtagagatttttcgatatttcggaaaattttgtaaaactgtaaatccgtaaaattttgctaaacatatattgtgttgttttggtaagtattgacccaagctatcacctcctgcagtatgtcgtgctaattactggacacattgtataacagttccactgtattgacaataataaaacctcttcattgacttgagaatgtctggacagatttggctttgtatttttgatttacaaatgtttttttggtgatttacccatccaaggcattctccaggatggttgtggcatcacgccgattggcaaacaaaaactaaaatgaaactaaaaagaaacagtttccgctgtcccgttcctctagtgggagaaagcatgaagtaagttctttaaatattacaaaaaaattgaacgatattataaaattctttaaaatattaaaagtaagaacaataagatcaattaaatcataattaatcacattgttcatgatcacaatttaatttacttcatacgttgtattgttatgttaaataaaaagattaatttctatgtaattgtatctgtacaatacaatgtgtagatgtttattctatcgaaagtaaatacgtcaaaactcgaaaattttcagtataaaaaggttcaactttttcaaatcagcatcagttcggttccaactctcaattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaggatctaattcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggctcgaagccggtcgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctccaggacggctccttcatctacaaggtgaagttcatcggcgtgaacttcccctccgacggccccgtaatgcagaagaagactatgggctgggaggcgtccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttagatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaacggagtactgtcctccgagcggagtactgtcctccgagcggagtactgtcctccgagcggagtactgtcctccgagcggagtactgtcctccgaggcaagacccttcctctatataaggaagttcatttcatttggagaggaatgtcctccgactcgtccaagatcaagaggaagcggaaccgcatcccgctcagctgcaccatctgccggaagaggaaggtcaagtgcgacaagctcaggccgcactgccagcagtgcaccaagaccggggtggcccacctctgccactacatggagcagacctgggccgaggaggccgagaaggagttgctgaaggacaacgagttgaagaagctcagggagcgcgtgaagtccttggagaagaccctctccaaggtgcactcctccccgtcgtccaactccacggcccccccgaccgacgtcagcctgggggacgagctccacttagacggcgaggacgtggcgatggcgcatgccgacgcgctagacgatttcgatctggacatgttgggggacggggattccccggggccgggatttaccccccacgactccgccccctacggcgctctggatacggccgacttcgagtttgagcagatgtttaccgatgcccttggaattgacgagtacggtggggaaggaagggggtcactattgacgtgtggtgatgtggaggagaacccaggcccaatgagagtcaaaacctttgtgatcttgtgctgcgctctgcaggtgagttaattattttactattatttcagaaggtggccagacgatatcacgggccacctgataataagtggtcgccaaaacgcacagatatcgtaaattgtgccatttgatttgtcacgcccgggggggctacggaataaactacatttatttatttaaaaaatgaaccttagattatgtaacttgtgatttatttgcgtcaaaagtaggcaagatgaatctatgtaaatacctgggcagacttgcaatatcctatttcaccggtaaatcagcattgcaatatgcaatgcatattcaacaatatgtaaaacaattcgtaaagcatcattagaaaatagacgaaagaaattgcataaaattataaccgcattattaatttattatgatatctattaacaattgctattgcctttttttcgcaaattataatcattttcataacctcgaggtagcattctgttacattttaatacattggtatgtgattataacacgagctgcccactgagtttctcgccagatcttctcagtgggtcgcgttaccgatcacgtgatagattctatgaagcactgctcttgttagggctagtgttagcaaattctttcaggttgagtctgagagctcacctacccatcggagcgtagctggaataggctaccagctaataggtagggaaaacaaagctcgaaacaagctcaagtaataacaacataatgtgaccataaaatctcgtggtgtatgagatacaattatgtactttcccacaaatgtttacataattagaatgttgttcaacttgcctaacgccccagctagaacattcaattattactattaccactactaaggcagtatgtcctaactcgttccagatcagcgctaacttcgattgaatgtgcgaaatttatagctcaatattttagcacttatcgtattgatttaagaaaaaattgttaacattttgtttcagtatgtcgcttatacaaat

SEQ ID NO: 28 - protein, 225 aa, Artificial Sequence
  Synthetic Construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFL

SEQ ID NO: 29 - protein, 173 aa, Artificial Sequence; identical to SEQ ID NO: 20
  Synthetic Construct

SEQ ID NO: 30 - protein, 18 aa, Artificial Sequence
  Synthetic Construct
EGRGSLLTCGDVEENPGP

(19) United States
(12) Patent Application Publication
(10) Pub. No.: US 2020/0325484 A1
(43) Pub. Date: Oct. 15, 2020

(54) Enhancing gene expression by linking self-amplifying transcription factor with viral 2A-like peptide

(71) Applicant: Delin Liang, Thousand Oaks, CA (US)

(72) Inventor: Delin Liang, Thousand Oaks, CA (US)

(73) Assignee: Delin Liang, Thousand Oaks, CA (US)

(21) Appl. No.: 16/382,210

(22) Filed: Apr. 12, 2019

(51) Int. Cl. C12N 15/63; C12N 15/81; C12N 15/86

(52) U.S. Cl. CPC C12N 15/635; C12N 15/86; C12N 15/81

(57) ABSTRACT

The invention describes a nucleic acid system, named as “2A-transcription amplifier”, for enhancing gene expression by linking a gene of interest (GOI) to a self-amplifying transcription factor with a viral 2A-like peptide. The system comprises an upstream activation sequence (UAS) at upstream promoter region and another sequence encoding a specific transcription factor (TF), a viral 2A-like peptide, and a gene of interest (GOI). The said compositions are operably linked in a way that the initially expressed TF protein binds the UAS region and promotes more TF and GOI co-expression. The viral 2A-like peptide separates the co-expressed TF and GOI protein during protein translation by the mechanism of ribosomal skidding. The system creates a transcription amplification loop that can be employed for enhancing expression of exogenous or endogenous gene of interest (GOI) in eukaryotic cells, tissues or whole organisms.

### TECHNICAL FIELD

This invention relates to the field of molecular biology. More specifically, the present invention pertains to compositions and methods of enhancing gene expression in eukaryotic cells and organisms.

### INCORPORATION-BY-REFERENCE OF SEQUENCE LISTING

The accompanying file, named Noahgen20190316SL.txt is 42 KB. The file can be accessed using Microsoft Word on a computer that uses Windows OS.

### BACKGROUND

Advances in molecular biology have offered many opportunities to develop genetically modified cells and organisms with commercially desirable characteristics or traits. Proper expression levels for a target gene, or gene of interest (GOI) in genetically modified cell or organism would be helpful in achieving this goal. However, despite the availability of many molecular tools, genetic modifications of host cells and organisms are often constrained by insufficient expression levels or uncontrolled expression of the GOI. There is always an unsatisfied goal to achieve the high expression of GOI in host cells and organisms.

In eukaryotic cells, gene expression is regulated on different levels including mRNA transcription, mRNA stability, protein translation and protein stability. Enhancing mRNA transcription is one of the most effective ways to enhance the expression level. Using a strong promoter is the most common technique for increasing transcription. Animal constitutive promoters of cytomegalovirus (CMV), eukaryotic translation elongation factor 1 α (EF1 α) and actin promoters have been identified and are broadly used in biotech protein expression systems. Plant constitutive promoters of cauliflower mosaic virus (CaMV) 35S, maize polyubiquitin and actin have been identified and are broadly used in transgenic plants. Yeast constitutive promoters of elongation factor 1-alpha-A (TEF1a) and glyceraldehyde-3-phosphate dehydrogenase (GPD) have been identified and are broadly used in biotech protein expression systems. However, these strong constitutive promoters are still not strong enough for some biotech applications like, for example, the industrial production of food and medically important proteins.

It has been shown that increased levels of specific transcriptional factors (TF) can be employed to increase the expression of a gene of interest (GOI). Schwechheimer described a gene expression feedforward loop system in which an upstream activation sequence (UAS) is operably linked to a transcription factor (TF) and a gene of interest (GOI) in each expression cassette, respectively (Schwechheimer et al., 2000). In this system, the small amount TF that is initially expressed binds the UAS to activate the further expression of both TF and GOI protein. This system is a self-amplifying transcriptional enhancing system with two cassettes expressing TF and GOI, respectively. Each cassette has its own promoter, coding region, and terminator. This two-cassette system, however, not only increases the difficulty for vector construction and transformation, but also requires a large cloning capacity for its plasmid or viral vectors.

### SUMMARY

This section provides a general summary of the invention, and is not comprehensive of its full scope or all of its features. In addition to the illustrative embodiments and features described herein, further aspects, embodiments, objects and features of the application will become fully apparent from the drawings and the detailed description and the claims.

This invention relates to methods of gene expression in eukaryotic cell systems. Specifically, this invention discloses a nucleic acid system wherein gene expression is enhanced to higher levels than that of prior art. More specifically, the nucleic acid system comprises one promoter region, one protein-coding region, and one terminator region, from 5′ to 3′ nucleic acid direction. The promoter region comprises an upstream activation sequence (UAS) and one minimal or intact promoter. The protein-coding region comprises a nucleic acid sequence encoding a specific transcription factor (TF) and a gene of interest (GOI), wherein TF and GOI are operably linked to each other with a nucleic acid sequence encoding a viral 2A-like peptide. The minimal promoter or intact promoter can initiate the expression of both the transcription factor (TF) and the gene of interest (GOI). The 2A-like peptide separates the transcription factor (TF) and gene of interest (GOI) proteins during protein translation by the mechanism of ribosomal skidding. The expressed transcription factor (TF) protein then binds the UAS specifically and further activates or promotes the expression of the transcription factor (TF) and the gene of interest (GOI). The more the transcription factor (TF) and the gene of interest (GOI) are expressed, the stronger the system's gene expression will be, until the system reach an intrinsic cellular gene expression maximum capacity. Thus, the system, named “2A-transcription amplifier” herein, is a self-amplifying gene expression system, in which transcription factor (TF) creates a self-amplifying positive feedback loop. The expression of the gene of interest (GOI) can reach higher levels than prior art.

The present disclosure relates a kind of viral 2A-like peptide that mediates “cleavage” of polypeptides during translation in eukaryotic cells. The 2A-like peptides separate the co-expressed transcription factor (TF) and gene of interest (GOI) protein during protein translation. This allows the 2A-transcription amplifier to be simplified as one nucleic acid sequence, or more specifically, one expression cassette. In other systems or prior art, an individual protein is normally cloned and expressed in each cassette that comprises a promoter, protein-coding region, and terminator. The present disclosure involves GOI and TF in only one expression cassette, which is small in terms of DNA size and makes DNA cloning easy in most vectors without exceeding the vectors' capacity. Compared with multiple UAS sequences in different cassettes in prior art, the present disclosure involves only one UAS in one cassette. There is no other UAS in other expression cassettes to compete for binding with the transcription factor (TF). Thus, 2A-transcription amplifier is more efficient in its function compared with other systems in this regard.

In certain embodiments, the 2A-transcription amplifier is constructed in a plasmid or DNA viral vector, which is maintained in eukaryotic cells or tissues as an episomal replicating element. In other embodiments, the 2A-transcription amplifier is integrated into a eukaryotic genome by transgenic approaches. While a gene of interest (GOI) is exogenous in most applications, a GOI can be endogenous in certain embodiments. The disclosure also includes that the 2A-transcription amplifier is employed to enhance an endogenous GOI expression by precise genome editing techniques.

The disclosure further includes the self-amplifying, everlasting and non-stopping expression nature of the 2A-transcription amplifiers. When combined with a tissue-specific promoter or inducible promoter, 2A-transcription amplifier provides expression systems with different enhanced expression levels with different temporal and spatial patterns.

### DESCRIPTION OF DRAWINGS

FIG. 1. Schematic presentation of a 2A-transcription amplifier, in which initially expressed transcription factor (TF) binds upstream activation sequence (UAS) to further amplify the expression of both the TF and gene of interest (GOI). A GOI sequence can be operably linked to either the N-terminus (Panel A) or C-terminus (Panel B) of a 2A-peptide sequence.

FIG. 2. A flow chart of the application of a 2A-transcription amplifier for enhancing an endogenous gene expression in a eukaryotic genome, indicating that a UAS-TF-2A fragment (Panel A) is integrated into a target genome (Panel B) through homology-dependent repairing (HDR) mechanism (Panel C).

FIG. 3. A yeast plasmid vector map with 2A-transcription amplifier for insulin production.

FIG. 4. A lentivirus vector map with a 2A-transcription amplifier for preproinsulin expression.

FIG. 5. A map of a donor vector fragment with a 2A-transcription amplifier for enhancing an endogenous silkworm Fib-H gene expression.

### DETAILED DESCRIPTION

### Definitions

Unless otherwise defined, all technical and scientific terms used herein have the same meaning as commonly understood by one of ordinary skill in the art to which this invention belongs.

The term “coding sequence”, or “coding region” refers to a nucleic acid sequence that once transcribed and translated produces a protein, for example, in vivo, when placed under the control of appropriate regulatory elements. A coding sequence as used herein may have a continuous open reading frame (ORF) or may have an ORF interrupted by the presence of a viral 2A-like peptide sequence.

The term “expression”, as used herein, refers to the process by which a polypeptide is produced based on the nucleic acid sequence of a gene. The process includes both transcription and translation. When two proteins or elements are “co-expressed”, they are induced at the same time and repressed at the same time. The levels at which two proteins are co-expressed need not been the same for them to be co-expressed. An “expression cassette” normally includes one promoter, one coding region and one terminator herein.

“DNA” refers to deoxyribonucleic acid. As used herein, “DNA”, “nucleotide sequence”, “nucleic acid sequence,” “nucleic acid,” or “polynucleotide,” refers to a deoxyribonucleotide in either single- or double-stranded form, and unless otherwise limited, encompasses known analogs of natural nucleotides that hybridize to nucleic acids in a manner similar to naturally-occurring nucleotides. Nucleic acid sequences can be, e.g., prokaryotic sequences, eukaryotic cDNA sequences from eukaryotic mRNA, genomic DNA sequences from eukaryotic DNA (e.g., mammalian DNA), and synthetic DNA, but are not limited thereto.

“DNA binding domain”, or “DBD”, refers to an independently folded protein domain that contains at least one structural motif that recognizes DNA sequence. A DBD can recognize a specific DNA sequence (a recognition sequence) or have a general affinity to DNA. Unless specifically mentioned, only specific DNA-binding is discussed in the invention.

“Gene of interest”, or “GOT”, herein refers to a nucleic acid fragment that encodes a target protein. Unless otherwise specified, a GOI herein refers only protein-coding gene that is transcribed by eukaryotic RNA polymerase II (RNAP II or Pol II). GOI herein refers to the coding region but not untranslated region (UTR). GOI can be a wide variety of heterologous sequences including, but not limited to, for example, sequences which encode growth factors, cytokines, chemokines, lymphokines, toxins, prodrugs, antibodies, antigens, ribozymes, as well as antisense sequences. A GOI herein can be endogenous or exogenous to a genome, may or may not include introns.

The term “operably linked” refers to positioning of a regulatory region and a sequence to be transcribed in a nucleic acid so as to control transcription or translation of such a sequence. In one example, a UAS is operably-linked to a protein-coding sequence with a core (basal) promoter in the middle. The UAS region can be immediately upstream of the basal promoter. The UAS can also be positioned as much as about 2,000 nucleotides upstream of the transcription start site. In another example, a transcription factor is operably-linked to a gene of interest (GOT) with viral 2A-like peptide in middle, the junction must be designed in such a way that ribosome skipping occurs correctly to produce a correct protein. “Unlinked” means that the associated genetic elements are not closely associated with one another and the function of one does not affect the other.

“Terminator” herein refers to a DNA sequence downstream of, or 3′ to, a coding sequence that causes RNA polymerase II to stop transcription. The terminator sequence can include a polyadenylation sequence. A terminator and a polyadenylation signal are used interchangeably herein.

“Transformation” refers to any a process by which nucleic acids are inserted into a recipient cell to effect change. Transformation may rely on known methods for the insertion of foreign nucleic acid sequences into a eukaryotic host cell. In mammalian cells, transformations can be accomplished by a variety of well-known methods, including, for example, electroporation, calcium phosphate mediated transfection, DEAE dextran mediated transfection, a biolistic method, a lipofectin method, and the like. In yeast or other fungi, transformation can be accomplished with LiOAc, protoplast, or electroporation method. In plant, transformation can be accomplished with agrobacterium or gene gun method. In insect, microinjection is the most popular transformation method.

“Upstream activation sequence” or “UAS ” refers to a nucleotide sequence that binds specifically with a corresponding transcription factor to activate the transcription of a gene. The upstream activation sequence is located “upstream” or 5′ to the coding region of a polynucleotide.

### 2A-Transcription Amplifier

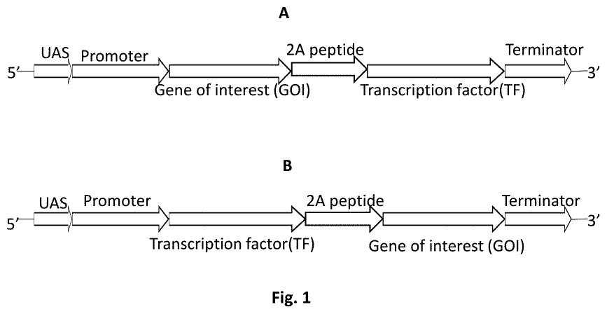

The invention describes a nucleic acid sequence system, named “2A-transcription amplifier” herein, for enhancing the expression of a gene of interest (GOI) by linking itself to a transcription factor with a viral 2A-like peptide. The 2A-transcription amplifier comprises an upstream activation sequence (UAS) at promoter region and a downstream sequence encoding a specific transcription factor (TF), a viral 2A-like peptide, and a gene of interest (GOI) (FIGS. 1A and 1B). The viral 2A-like peptide separates the co-expressed TF and GOI protein during protein translation by the mechanism of ribosomal skidding. The TF comprises at least one transcription activation domain (AD) and at least one DNA binding domain (DBD), which, upon expression, binds the UAS specifically and amplifies the expression of the TF and GOI. The compositions are operably linked in a way that the initially expressed TF protein binds the UAS region and promotes more TF and GOI co-expression. The 2A-transcription amplifier creates a positive transcription feedback loop that can be employed for enhancing gene of interest (GOI) expression in eukaryotic cells, tissues and whole organisms.

### Viral 2A-Like Peptides

Viral 2A-like peptides were initially identified in foot and mouth disease virus (FMDV), a member of the Picornaviridae family. “Viral 2A-like peptides” is interchangeably used as “2A-like peptides”, “2A-like” oligopeptides, “2A self-cleaving peptides”, or “2A peptides” herein. They allow multiple discrete proteins to be synthesized from a single strand of virus RNA, which also functions as a messenger RNA (mRNA) in the infected cell. The designation “2A” refers to a specific viral protein of the viral genome and different viral 2As have generally been named after the virus they are derived from. Viral 2A-like peptides include but not limit to a group consisting of a foot-and-mouth disease virus (FMDV) 2A (F2A, SEQ ID NO: 1), a Thosea asigna virus 2A (T2A, SEQ ID NO: 2), a porcine teschovirus-1 2A (P2A, SEQ ID NO: 3), an equine rhinitis A virus (ERAV) 2A (E2A, SEQ ID NO: 4), a Bombyx mori cytoplasmic polyhedrosis virus (BmCPV) 2A (BmCPV2A, SEQ ID NO: 5), a Bombyx mori infectious flacherie virus (BmIFV) 2A (BmIFV2A, SEQ ID NO: 6), and a combination thereof. Viral 2A peptides are 18-22 amino-acid (aa)-long virus-encoded oligo-peptides that mediate “cleavage” of polypeptides during translation in eukaryotic cells (Ahier 2014). Viral 2A-like peptides share an “Asp-Val/Ile-Glu-Xaa-Asn-Pro-Gly-Pro” consensus motif, wherein Xaa is any amino acid (SEQ ID NO: 7) (Donnelly et al., 2001).

Picornaviruses are not the only species possessing a sequence that carries out this function. 2As have been found in a substantial variety of genomes, such as unicellular organisms of Trypanosoma (Odon et al., 2013) and purple sea urchin Strongylocentrotus purpuratus (Roulston et al., 2016). As the number of genomes sequenced increases ever more rapidly due to advances in sequencing technology, more and more 2As are being discovered. From this ever-expanding library of 2As, it has now become possible to carry out comparisons between sequences and attempt to determine the essential components that confer their function. A number of amino acids at specific positions in the sequences are conserved, and as such represent the 2A signature. This signature is suspected to be the region that binds to the ribosome exit tunnel and cause the skipping mechanism. Identification of the 2A signature has made the discovery of additional 2As significantly easier, as a species' genome can be systematically searched for the presence of the defining series of amino acids. It is thus conceivable that more naturally existing or synthetic 2A-like peptides with the consensus motif can be used in the 2A-transcription amplifier.

Despite the initial “self-cleavage” theories for the mechanism of action of 2A, it has since been shown to operate in a completely different mode, termed “ribosome skipping”. This mechanism does not involve the synthesis of a polyprotein followed by cleavage, but instead the discrete synthesis of the constituent proteins. In the case of a single strand of mRNA that encodes both transcription factor (TF) and gene of interest (GOI) separated by the 2A sequence, the ribosome synthesizes the “first protein” as normal and then continues to add the 2A sequence onto the end. Once 2A produced, this sequence of amino acids interacts with the exit tunnel of the ribosome and prevents further elongation. To remove this blockage, the protein is released from the ribosome as if it had encountered a stop codon, and protein synthesis can resume on the “second protein” downstream of the 2A. This is a translational control of protein expression, rather than the more commonly observed transcriptional control.

Viral 2A-like peptides sequences (consensus sequence “Asp-Val/Ile-Glu-X-Asn-Pro-Gly-Pro”, wherein Xaa is any amino acid), during translation, force the ribosome to skip from the underlined Gly to the underlined Pro codon without forming a glycyl-prolyl peptide bond at the C-terminus of the 2A. (Donnelly et al., 2001). Consequently, the nascent translation product (herein “first protein”) is released after the addition of the glycine residue and a new, independent protein chain (herein “second protein”) is begun with the proline residue. The said first protein bears “Asp-Val/Ile-Glu-X-Asn-Pro-Gly” amino acid residues at its C-terminus, while the said second protein bears a proline residue at its N-terminus. It was shown that in some cases the “first protein” is expressed at an amount that is greater than the “second protein” in such a translation system. Besides, while a large amount of protein tolerates a few extra residues at their termini, some protein products may be sensitive to extra amino acids residues at N-terminus or C-terminus for a normal function. Based on these considerations, a gene of interest (GOI) can be designed at either the “first protein” (FIG. 1A) or “second protein” position (FIG. 1B).

In some embodiments, co-translational signal sequences are included for the “first protein” and “second protein”, normally at the N-terminus ends, respectively. This allows both “first protein” and “second protein” to be directed to a different cell compartment, respectively (Roulston et al, 2016). Thus, while the transcription factor (TF) is directed to nucleus by its nucleus localization sequence (NLS), the co-translated protein of the gene of interest (GOI) can be directed to another target compartment or organelle such as nucleus, cytosol, endoplasmic reticulum, Golgi apparatus, vacuoles, plasma membrane, chloroplast, or mitochondria. In some embodiments, multiple genes of interest (GOI) can be operably linked to each other with same or different 2A peptides.

Transcription Factor

Transcription factors (TFs) herein refers to a big family of proteins that are modular in structure, containing both DNA-binding domain (DBD), trans-activating domain (AD) and nuclear localization sequence (NLS). Unless specified, NLS herein is included as part of selected DBD or AD domain in each transcription factor (TF). In some embodiments, the transcription factor (TF) of the 2A-transcription amplifier is selected from naturally-occurring proteins such as Gal4, Hap1, QF, c-Myc, c-Fos, c-Jun, CREB, cEts, GATA, c-Myb, MyoD, and NF-κB, Hif-1, and TRE. In other embodiments, a transcription factor is a synthetic protein with DBD and AD domains from difference protein sources.

DNA-Binding Domain

A DNA-binding domain (DBD) is an independently folded protein domain that contains at least one structural motif that recognizes and binds DNA sequences. There are different types of DBD founds across prokaryotic to eukaryotic organisms. The types of DBD include, but not limited to, helix-turn-helix domain, zinc finger domain, Leucine zipper domain, winged helix, winged helix-turn-helix domain, helix-loop-helix domain, HMG-box, and Wor3 domain, and ribonucleoprotein (RNP) domain. Gal4 DBD has been used in plant, insect and mammalian cells successfully. Hap1 DBD has been used in plants successfully. LexA DBD has been used in numerous eukaryotic hosts including fungi, plant and animals successfully. Neurospora crassa QF transcription factor DBD has been used in insects successfully. Preferred DBDs that can be used in the invention include, but are not limited to, LexA, Gal4, Hap1, Adr1, Ace2, Cup2, Bas1, Gcn4, Swi5, Pho4, LacI. QF1, SP1, AP-1, C/EBP, Heat shock factor, ATF/CREB, c-Myc, Oct-1, NF-1, tetracyclin repressor, and ZFHD-1.

In some embodiments, a selected transcription factor is required to have no negative effects on target cell or organism. More specifically, a selected transcription factor is required not to interfere other unrelated, off-target genes. Thus, a DNA-binding domain (DBD) for the 2A-transcription amplifier prefers not to be native to their target cells or organisms to avoid potential host growth side effects. For example, the DBD of the yeast transcription factor Gal4 is suitable for use in mammalians, insects and plants. There are no endogenous genes of mammalian, insect or plant appearing to be the target of exogenous Gal4 regulation. A 2A-transcription amplifier with gal4 DBD may not be suitable for yeast hosts for physiology studies. The disclosure includes amino acid sequences for some most often used DNA-binding domains (DBDs). They are yeast Gal4 (SEQ ID NO: 8); yeast Hap1 (SEQ ID NO: 9) and E. coli LexA (SEQ ID NO: 10).

Activating Domain

Activating domain (AD) of a transcription factor is the domain that binds other proteins such as transcription coregulators to initiate a gene's transcription. In general, there are four classes of activating domain (AD) (Mitchell et al., 1989): a) acidic domains, rich in D and E amino acids; b) glutamine-rich domains, with multiple repetitions like “QQQXXXQQQ”, wherein Q is glutamine and X is any amino acid; c) proline-rich domains, with repetitions like “PPPXXXPPP”, wherein P is proline and X is any amino acid; d) isoleucine-rich domains, with repetitions “IIXXII”, wherein I is isoleucine and X is any amino acid. Proteins containing ADs include Gal4, Gcn4, Oaf1, Leu3, Rtg3, Pho4, Gln3 in yeast; THM18, Dof1, bZIP and maize transcriptional activator C1 in plant; and steroid hormone receptors, heat shock transcription factors, glucocorticoid receptor, NFKBp53, NFAT, and NF-κB in mammals; TAT and VP16 in in viruses. Many ADs are as short as 9 amino acids. Nine-amino-acid transactivation domain (9aa AD) is a domain common to a large superfamily of eukaryotic transcription factors represented by Ga14, Gln3, Gcn4, Oaf1, Leu3, Rtg3, and Pho4 in yeast and by VP16, p53, NFAT, and NF-κB in mammals. When selecting an AD for the 2A-transcription amplifier described in this invention, small AD sequence size, strong activation activity and no-negative effects on host cell's normally growth are among the factors to be considered. Preferred transcriptional activation domains include but are not limited to the VP16, B42, Gal4, Hap1, Add Ace2, Cup2, Bas1, Gcn4, Swi5, Pho4, and Ste 12.

The disclosure also includes amino acid sequences for some most often used transcriptional activation domains (ADs). They are amino acid sequence of transcriptional activation domain (AD) of herpes simplex virus protein VP16 (SEQ ID NO: 11) and Zea mays protein C1 (SEQ ID NO: 12).

Upstream Activation Sequence

A DBD can bind a specific DNA sequence (a recognition sequence). Gal4 binds to DNA sequences with the consensus of 5′-CGG-N11-CCG-3′. N herein is any of the nucleotide A, T, G, or C. LexA binds to DNA sequences with the consensus of 5′-TACTG-(TA)5-CAGTA-3′. Hap1 binds to DNA sequences with the consensus of 5′-CGG-N3-TANCGGN-3′. Neurospora crassa QF transcription factor binds to DNA sequences with the consensus of 5′-GGRTAARYRYTTATCC-3″ (R is A/G, Y is C/T). Followings are more DBD recognition sequences with protein or domain names in front of them, respectively: SP1(5′-GGGCGG-3′); AP-1 (5′-TGA(G/C)TCA-3′); C/EBP (5′-ATTGCGCAAT-3′); Heat shock factor (5′-NGAAN-3′); ATF/CREB (5′-TGACGTCA-3′); Basic helix-loop-helix of c-Myc (5′-CACGTG-3′); Helix-turn-helix of Oct-1 (5′-ATGCAAAT-3′); NF-1(5′-TTGGC-N5-GCCAA-3′); Lac operon (5′ -AATTGTGAGCGCTCACAATT-3′); AraC (5′-TATGGATAAAAATGCTA-3′).

A nucleic acid sequence with the DBD consensus recognition sites can be located at the upstream of a gene coding region to form an upstream activation sequence (UAS). There can be one to multiple copies of the UAS in tandem. The copy number of UAS can be up to but not limit to twenty. Transcription activity normally increases along with the increasing of UAS copy number. However high copy number increases the cloning difficulty and instability of the sequence. Normally five-ten copies of UAS are used in tandem. For example, five copies of UAS (5×UAS) is used in this invention. SEQ ID NO: 13 is the nucleic acid sequence of 5×UAS for Gal4. SEQ ID NO: 14 is the nucleic acid sequence of 5×UAS for Hap1. SEQ ID NO: 15 is the nucleic acid sequence of 5×UAS for LexA.

In some embodiments, the nucleic acid sequences of the said coding region of TF, GOI and 2A-like peptide are codon-optimized. The codon usage of the coding sequence can be adjusted to achieve a desired property, for example mRNA stability and high levels of expression in a specific species. Software tools for codon optimization of a gene to different species are available from companies such as Noahgen, Integrated DNA Technologies (IDT), GenScript, and Thermofisher Scientific.

Promoter for the 2A-Transcription Amplifier

“Promoter” refers to a nucleic acid sequence at the 5′ end of a gene or polynucleotide which directs the initiation of transcription. In general, a coding sequence is located 3′ to a promoter sequence. “Promoter” includes a minimal promoter that is a short DNA sequence comprising a TATA-box and other sequences that serve to specify the site of transcription initiation, to which regulatory elements are added for control of expression. This type of promoter sequence consists of proximal and more distal upstream elements, the latter elements often referred to as enhancers. Accordingly, an enhancer is a DNA sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions.

In some embodiments, there is only a minimal promoter, basal promoter, or TATA-box in the downstream of the said UAS for the 2A-transcription amplifier. “Minimal promoter”, “basal promoter”, and “TATA-box” are used interchangeably herein. Basal promoter is the minimal sequence necessary for assembly of a transcription complex required for transcription initiation. Basal promoters frequently include a “TATA-box” element that may be located between about 15 and about 35 nucleotides upstream from the site of transcription initiation. The minimal promoter for the 2A-transcription amplifier can be selected from a group consisting of nucleic acid sequence of minimal 35S promoter of cauliflower mosaic virus (CaMV) (SEQ ID NO:16) , nucleic acid sequence of heat shock protein 70 basal promoter of Drosophila melanogaster (SEQ ID NO:17), and nucleic acid sequence of cytomegalovirus (CMV) minimal promoter (SEQ ID NO:18). In most cases, a translation start codon (ATG) is avoided in the UAS or minimal promoter region. When the self-amplifying 2A-transcription amplifier is introduced into a cell, the minimal promoter will initiate the basal expression of gene of interest (GOI) as well as transcription factor (TF), wherein TF will then bind to the UAS and initiate the further transcription (FIGS. 1A and 1B). The more transcription factor is expressed, the stronger the further expression of both GOI and TF can be achieved. The amplifying loop will not stop until reaching the maximum capacity of cell gene expression. The above well-characterized minimal promoters are very short in nucleic acid sequences and therefore easy and flexible for vector DNA cloning.

In some embodiments, an intact (or full) promoter is located at the downstream of the said UAS in the 2A-transcription amplifier. The intact promoter can be constitutive or tissue specific, strong or weak, temporal specific or spatial specific. A constitutive promoter is active in all circumstances in an organism, while others are regulated, becoming active in only certain cells only in response to specific stimuli. A tissue-specific promoter is a promoter that has activity in only certain cell types.

In some embodiment, the promoter is an intact constitutive promoter, whether it is strong or weak, the 2A-transcription amplifier will amplify the transcription and express more GOI product than using promoter alone without UAS. Useful promoters that may be used in the invention include, but are not limited to, eukaryotic elongation factor 1-alpha 1 (EF1a) promoters, polyubiquitin promoters, actin promoters and tubulin promoters from eukaryotes, cytomegalovirus (CMV) promoter, SV40 virus early promoter, agrobacterium nopaline synthetase (nos) promoter, cauliflower mosaic virus (CaMV) 35S promoter, fungi glyceraldehyde-3-phosphate dehydrogenase promoter. When selecting a promoter for the 2A-transcription amplifier, both promoter activity and sequence length need to be considered. In general, a small size promoter that is no more than 1kb is suitable for the 2A-transcription amplifier.

In some embodiments, the promoter is a tissue-specific promoter, the transcriptional self-amplifying will not stop after promoter stops but continues the amplification process unless the whole gene expression system is turned down in the scenarios such as in a dormant plant seed or fungus spore. If the promoter is stringent specific, such an expression pattern is everlasting with a distinct start point, which is different from a constitutive promoter expression pattern which does not have a distinct start point. The everlasting and enhancing nature of the 2A-transcription amplifier will add new tools for gene regulation in genetic modified organism (GMO).

In some embodiments, the gene of interest (GOI) is a reporter gene such as a fluorescence protein or antibiotic resistance gene. There are some genes in eukaryotic organisms that are expressed only in transient and weak levels. It has been shown that a lot of genes were expressed transiently at early mammalian development stage. The expression of the genes and their effects on development are difficult to confirm and evaluate. The 2A-transcription amplifier of the invention can also be exploited to track or select cell lineages deriving from the specific tissue or cells.

In some embodiments, the promoter in a 2A-transcription amplifier is an inducible promoter. Some inducible promoter activity responds to chemical factors such as tetracycline, alcohol, galactose, lactose or lactose analog IPTG, steroid, oleic acid, ecdysone and estrogen. Some inducible promoter activity responds to chemical factors such as light, heat-shock, cold-shock. Similar to using other promoters, the expression levels will be amplified in the 2A-transcription amplifier after an inducible promoter initiates the expression of both transcription factor (TF) and gene of interest (GOI). The expression in the 2A-transcription amplifier will not stop even after the inducing factors disappear. Therefore, the amount of inducing chemicals can be reduced if necessary.

Gene of Interest (GOI) as Exogenous Gene

In some embodiments, a 2A-transcription amplifier is constructed into a DNA vector. A “vector” is a replicon, such as a plasmid or DNA virus, into which another DNA segment may be inserted so as to bring about the replication of the inserted segment. Vector backbones include, but not limit to, plasmids, BACs, YACs, PACs, baculoviruses, retroviruses, adenoviruses and adeno-associated viruses. The vector can contain sequences that facilitate recombinant DNA manipulations, including, for example, elements that allow propagation of the vector in a particular host cell (e.g., a bacterial cell, insect cell, yeast cell, or mammalian cell), selection of cells containing the vector (e.g., antibiotic resistance genes for selection in bacterial, plant, insect or mammalian cells), and cloning sites for introduction of reporter genes or the elements to be examined (e.g., restriction endonuclease sites or recombinase recognition sites).

Vectors have limitations in their size and their cloning capacity. Most general plasmids may be used to clone DNA fragments of up to 15 kb in size. Lentivirus vectors can package large DNA fragments with a sharp titer decline after 10 kb total proviral size. While artificial chromosomes such as BACs, YACs and PACs have relative larger cloning capacity, they are low copy vectors in hosts and difficult for cloning. Each 2A-transcription amplifier has only one promoter region, one coding region, and one terminator. The size of a 2A-transcription amplifier is 1-2 kb if not counting the gene of interest (GOI). The fragment is small enough to be cloned into most of vectors.

A 2A-transcription amplifier can be maintained in the vectors as a replicating epi-chromosomal plasmid or viral vector in a eukaryotic cell or organism. Such vectors include 2-micron plasmids and autonomously replicating sequence (ARS) plasmids in yeast cells, baculoviruses in insect cells, adenoviruses and adeno-associated viruses in mammalian cells. The 2A-transcription amplifier can also be integrated into the genome of a target eukaryotic cell or organism. Such vectors include integration vectors for yeast, retroviruses for mammalian cells, and agrobacterium Ti plasmids for plants.

DNA cloning techniques commonly known in the art can be found, e.g., in Ausubel et al. eds., 1995, “Current Protocols in Molecular Biology”, and in Sambrook et al., 1989, “Molecular Cloning: A Laboratory Manual”, Cold Spring Harbor Laboratory Press, NY. It should be noted that DNA synthesis and cloning of the 2A-transcription amplifier fragment and even the whole vector can be outsourced to biotech service companies such as Noahgen, Genscript, and Thermofisher.

Gene of Interest (GOI) as Endogenous Gene

Eukaryotic gene sizes in genome vary over a wide size range. Many genes include multiple introns and therefore may span a larger region. About 15% of human genome transcripts span greater than 100 kb of genomic sequence. For examples, human Caspr2 protein gene (CNTNAP2) spans 2.3 Mb of genomic sequence; human Titin protein, also known as Connectin, has the length of ˜27,000 to ˜33,000 amino acids (depending on the splice isoform). Many eukaryotic genes undergo alternative splicing and produce multiple gene products. It is thus importance to keep genomic non-coding region which includes intron regions in some biotech applications. Therefore, it is not amenable to clone and express these large genes as exogenous genes in a eukaryotic cell or organism. To enhance a large endogenous gene's expression, the 2A-transcription amplifier can be precisely integrated in front of the gene's coding region.

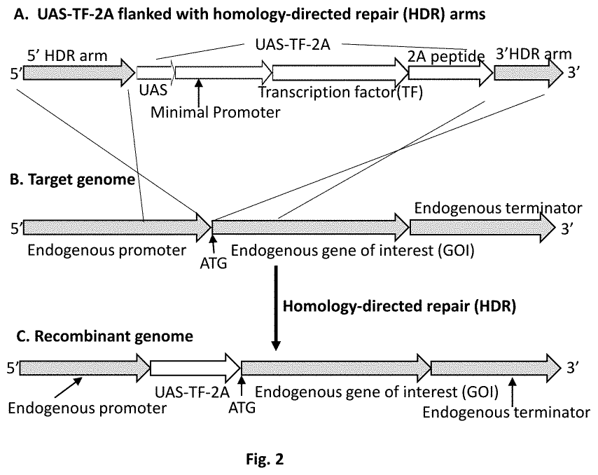

In certain embodiments, the 2A-transcription amplifier can be employed to enhance the expression of an endogenous gene of interest (GOI) in a eukaryotic cell, tissue or organism [FIG. 2]. To this end, a nucleic acid sequence of “UAS-minimal promoter-TF-2A peptide” (“UAS-TF-2A” in short) (FIGS. 2A and 2C) can be engineered and inserted into the upstream of the start codon (ATG) of an endogenous gene of interest (GOI) in a target genome (FIG. 2B). The precise integration can be achieved by homology-dependent repairing (HDR) mechanism in eukaryotic cells (Gaj et al., 2013). The “UAS-TF-2A” nucleic acid sequence is further flanked with recombination arms, which are promoter region and coding region of the endogenous GOI, respectively (FIG. 2A). The arm regions are normally one kilobase in length, respectively. A DNA fragment of “UAS-TF-2A” with franking homologous arms can be a sigle DNA fragment or a part of plasmid or viral vector, wherein the vector is also called donor vector for recombination. The donor fragment or vector can be transformed and integrated into a target genome through homology-dependent repairing (HDR). There is plenty of prior art on genetic transformation methods for eukaryotic cells or organisms, including fungi, plants, insects and animals. To promote efficient homology-dependent repairing (HDR), a DNA break (or gap) is generated around the start codon (ATG) site of the endogenous GOI (FIGS. 2B and 2C) by co-transformation the donor fragment or vector with designed enzymes such as CRISPR cas9, Talen or zinc-finger nuclease (Gaj et al., 2013). The modified region of the recombinant genome will be “endogenous promoter-UAS-TF-2A-endogenous gene of interest (GOI)-endogenous terminator” (FIG. 2C). The coding region of TF and endogenous GOI in the transformed genome is operably linked to each other with the 2A peptide nucleic acid sequence. The “UAS-minimal promoter” is operably linked to the upstream endogenous promoter region. In this system, the minimal promoter initiates the expression of TF and the endogenous GOI expression. The expressed TF binds the UAS region and promotes more expression of both GOI and TF, which creates a positive feedback loop.

In some embodiments, the efficacy of homology-dependent repairing (HDR) is too low to get a positive genome modification without a selection. To enhance the screening efficiency, a selection marker expression cassette can be linked immediately in front of the UAS-TF-T2A fragment. The marker cassette will not interfere the self-amplifying expression 2A-transcription amplifier in most cases. Furthermore, the selection marker expression cassette can be designed as excisable genetic fragment by flanking itself with specific enzyme recognition sites such as Cre-lox sites (Turan, S. et al., 2011), Flp-FRT sites (Rao M. R. et al., 2010) and Piggybac inverted terminal repeats (ITRs) (Li et al., 2013). The selection marker can also be excised efficiently with the designed specific enzymes such as CRISPR cas9, Talen and Zinc-finger nucleases (Gaj et al., 2013).

The self-amplifying expression 2A-transcription amplifier can be applied to most if not all endogenous protein-coding gene in a eukaryotic genome. The endogenous genomic genes of interest (GOI) can be commercial important genes encoding, for example, storage proteins in plant seeds and silk protein of silkworm. They can be medically important genes, such as insulin gene, erythropoietin (EPO) gene and insulin-like growth factor-1, that can be the target of gene therapy for gene enhancement purposes.

### EXAMPLES

### Example 1

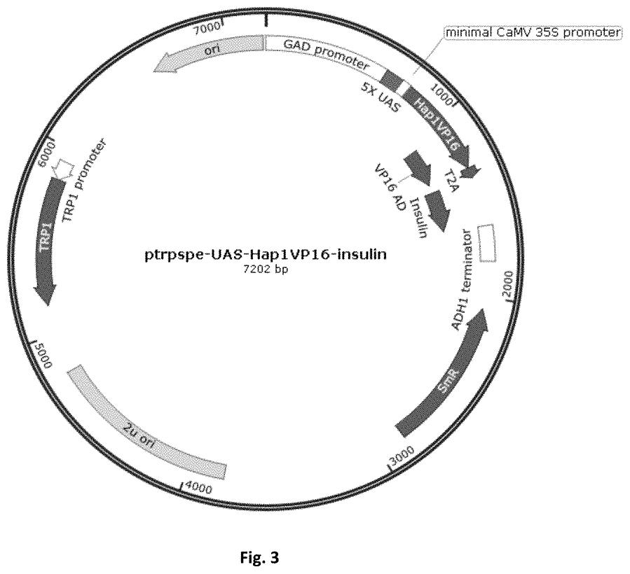

In one embodiment, a self-amplifying gene expression 2A-transcription amplifier was constructed into a yeast-E coli shuttle plasmid vector ptrpspe-UAS-Hap1VP16-insulin [FIG. 3]. The 2A-transcription amplifier comprises, from 5′ to 3′ nucleic acid direction, yeast GAP promoter, 5×UAS-minimal CaMV 35s promoter, transcription factor Hap1VP16, T2A peptide, insulin and ADH1 terminator. The nucleic acid sequence is disclosed as SEQ ID NO: 19. The plasmid vector also comprises E coli replication origin (ori) and spectinomycin resistance gene (SmR). The plasmid vector also comprises yeast 2-micron plasmid replication ori (2 μ ori) and selection marker Trp1.

The plasmid vector can be transformed into yeast Saccharomyces cerevisiae and expresses high yield of insulin protein. Yeast GAD promoter is constitutive promoter from yeast glyceraldehyde-3-phosphate dehydrogenase gene. Yeast plasmid can be transformed into yeast with LiOAc method (Liang et al., 2003). The transformed yeast can grow in YNB-trp medium. One liter of YNB medium contains 20 g glucose, 1.7 g yeast nitrogen base, 5 g ammonium sulfate, 0.6 g-trp amino acids dropout mix from Sigma-Aldrich. Once the plasmid is transformed into yeast host, yeast GAD promoter initiates the expression of Hap1VP16 -T2A peptide-insulin. During translation, T2A peptide separates Hap1VP16 and insulin protein by the mechanism of ribosomal skidding. The initially expressed transcription factor Hap1VP16 binds to the 5×UAS sequence and promotes more expression of Hap1VP16 as well as insulin. For secretory expression of insulin, a signal peptide can be further added immediately upstream of the insulin peptide sequence (Balschmidt et al., 2001).

### Example 2

In another embodiment, a self-amplifying gene expression 2A-transcription amplifier was constructed into a 3rd generation lentiviral vector pLenti-UAS-preproinsulin-Gal4VP16 [FIG. 4]. The 2A-transcription amplifier comprises, from 5′ to 3′ of nucleic acid direction, 5×UAS for Gal4 DNA-binding domain (DBD), CMV promoter, coding region of human preproinsulin-F2A peptide-Gal4 DBD-VP16 AD. The nucleic acid sequence is disclosed as SEQ ID NO: 20.

The 2A-transcription amplifier is flanked with “SV40 promoter-blasticidin (BSD)” marker expression cassette, viral RRE gene and psi packaging signal (HIV-1 Ψ), and lentiviral long terminal repeat (LTRs) sequences including HIV 5′ region (LTR) and 3′ LTR (AU3). Together with helper plasmids encoding Rev, Gag and Pol and vesicular stomatitis virus G (VSV-G) protein, transfection of Human embryonic kidney HEK293T cells with the lentiviral vector will produce VSV-G pseudotyped lentiviral virions. Unlike the HIV envelope, the VSV-G envelope has a broad cell host range extending the cell types that can be transduced by VSV-G-expressing lentiviruses (Joglekar et al., 2017).

Two days after transfection of HEK 293T cells, the cell supernatant contains recombinant lentiviral genome, which is used to transduce the mammalian target cells. Once in the target cells, the viral RNA is reverse-transcribed, imported into the nucleus and stably integrated into the host genome. One or two days after the integration of the viral RNA, the strong expression of the GOI insulin protein is detected and purified. In most cell types, CMV promoter and amplifier regulate strong expression of preproinsulin-F2A-Gal4VP16 expression. Expressed Gal4VP16 protein will then bind to 5XUAS and promote further expression of both preproinsulin and Gal4VP16, which creates an amplification loop. The pseudo-typed lentiviral virions can also further be employed as a gene therapy vector to enhance insulin expression in vivo. Insulin signal sequence at N-terminus of preproinsulin protein will be processed when mature insulin is secreted.

### Example 3

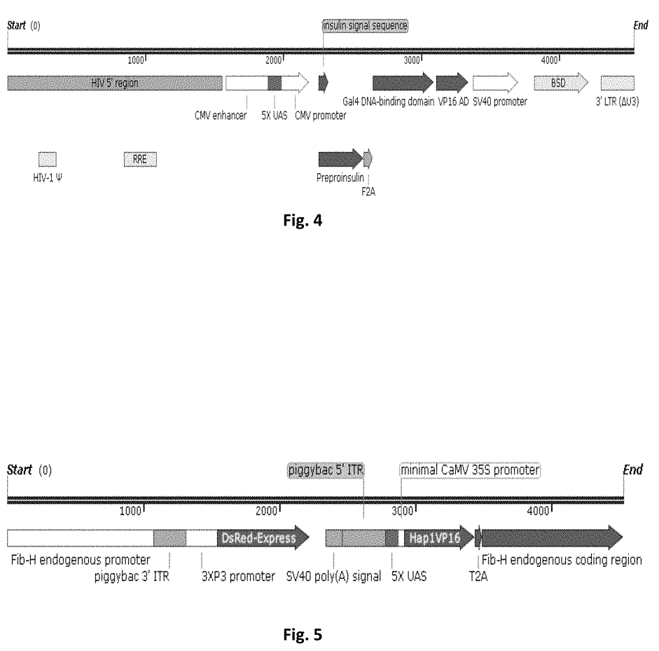

The self-amplification gene expression 2A-transcription amplifier is employed to enhance or the expression of endogenous fibroin heavy chain (Fib-H) protein in domestic silkworm (Bombyx mori) [FIG. 5]. Fibroin heavy chain is one of the major components of cocoon or silk, which is an important material for not only textiles and industrial applications but also biomaterials and cosmetics. There have been extensive efforts in enhancing silk protein synthesis. Fib-H coding region has many repeat motifs and is about 16 kb in length, which is not amenable for cloning.

To this end, a nucleic acid sequence of “5×UAS-minimal CaMV 35S promoter-Hap1VP16-T2A peptide” was engineered in a donor vector pleukan-Scarless-FibH. The nucleic acid sequence is flanked with two recombination arms, which are Fib-H endogenous promoter region and coding region, respectively. The arm region is normally one kilobase in length. For easy screening of transgenic positive individuals, a “3XP3 promoter-dsRed-SV40 polyadenylation” reporter cassette is also cloned into the vector. The reporter cassette is flanked with 5′ and 3′ piggybac inverted terminal repeats (ITRs) at each end, respectively, so that the marker can be excised by transposase after selection (FIG. 5). The nucleic acid sequence is disclosed as SEQ ID NO: 21.

The precise integration was achieved by homology-dependent repairing (HDR) mechanism in eukaryotic cells [FIG. 2]. The transformation method is the same as previously reported (Cui et al., 2018). To promote efficient HDR, a DNA break (or gap) is generated around the start codon (ATG) site of the endogenous Fib-H gene by CRISPR cas9. The target sequence of gRNA was disclosed as “ttgactctcatcttgagagt”. The purified DNA for donor vector, cas9 and guide RNA vector were mixed with appropriate ratio and microinjected into silkworm eggs. Biotech companies proving insect microinjection and CRISPR services include WellGenetics, Rainbow Transgenic Flies, and Genetic vision. G1 progenies with red fluorescence eyes were identified as positive transgenic silkworms. After crossing with a silkworm expressing piggybac transposase, the dsRed cassette as well as the flanking piggybac 5′ ITR and 3′ ITR were cut out seamless (Singh et al., 2015). The final transgenic silkworms thus have the gene structure of “endogenous Fib-H promoter-5×UAS-minimal promoter-Hap1VP16-T2A-endogenous Fib-H coding region-endogenous terminator”. The initially expressed Hap1VP16 binds the UAS region and promotes more expression of both Hap1VP16 and fibroin heavy chain, which creates an amplification loop.

All of the compositions and methods disclosed herein can be made and executed without undue experimentation in light of the present disclosure. It is to be understood that while the invention has been described in conjunction with the detailed description thereof, the foregoing description is intended to illustrate and not limit the scope of the invention, which is defined by the scope of the appended claims. More specifically, it will be apparent that certain agents which are both chemically and physiologically related may be substituted for the agents described herein while the same or similar results would be achieved. Although the invention has been described with reference to the above examples, it will be understood that modifications are encompassed within the scope of the invention.

### NON-PATENT CITATIONS

Ahier, A. et al., 2014. Simultaneous expression of multiple proteins under a single promoter in Caenorhabditis elegans via a versatile 2A-based toolkit. Genetics 196(3):605-613.

Balschmidt, P. et al., 2001. Expression of insulin in yeast: the importance of molecular adaptation for secretion and conversion. Biotechnology & genetic engineering reviews 18(1):89-121.

Boron, W. F. 2003. Medical Physiology: A cellular and molecular approach. Elsevier/Saunders. pp. 125-126.

Brent, R. et al., 1985. A eukaryotic transcriptional activator bearing the DNA specificity of a prokaryotic repressor. Cell. 43:729-736.

Schwechheimer, C. et al., 2000. Transactivation of a target gene through feedforward loop activation in plants. Funct Integr Genomics. 1(1):35-43.

Cui, Y. et al., 2018. New insight into the mechanism underlying the silk gland biological process by knocking out fibroin heavy chain in the silkworm. BMC Genomics.19:215

Donnelly, M. L. et al., 2001. The ‘cleavage’ activities of foot-and-mouth disease virus 2A site-directed mutants and naturally occurring “2A-like” sequences. J. Gen. Virol. 82: 1027-1041.

Gaj, T. et al., 2013. ZFN, TALEN and CRISPR/Cas-based methods for genome engineering. Trends Biotechnol. 31(7): 397-405.

Ha, N. et al., 1996. Mutations in target DNA elements of yeast HAP1 modulate its transcriptional activity without affecting DNA binding. Nucleic Acids Research 24 (8):1453-1459.

Joglekar, A. V. et al., 2017. Pseudotyped lentiviral vectors: one vector, many guises. Hum Gene Ther Methods. 28(6):291-301.

Li, X. at al., 2013. PiggyBac transposase tools for genome engineering. Proc Natl Acad Sci USA. 110(25): E2279-87.

Liang, D. et al., 2004. Site-directed mutagenesis and generation of chimeric viruses by homologous recombination in yeast to facilitate analysis of plant-virus interactions. Mol Plant Microbe Interact. 17(6):571-576.

Liu, Z. et al., 2017. Systematic comparison of 2A peptides for cloning multi-genes in a polycistronic vector. Sci Rep. 7(1):2193.

Mitchell, P. et al., 1989. Transcriptional regulation in mammalian cells by sequence-specific DNA binding proteins. Science. 245 (4916): 371-378.

Odon, V. et al., 2013. APE-type non-LTR retrotransposons of multicellular organisms encode virus-like 2A oligopeptide sequences, which mediate translational recoding during protein synthesis. Mol Biol Evol. 30(8):1955-65.

Piskacek S. et al., 2007. Nine-amino-acid transactivation domain: establishment and prediction utilities. Genomics. 89 (6): 756-768.

Rao, M. R. et al., 2010. FLP/FRT recombination from yeast: application of a two gene cassette scheme as an inducible system in plants. Sensors (Basel). 10(9): 8526-8535.

Roulston, C. et al., 2016.‘2A-Like’ Signal sequences mediating translational recoding: a novel form of dual protein targeting. Traffic. 17(8): 923-939.

Singh, A. M. et al., 2015. Gene editing in human pluripotent stem cells: choosing the correct path. J Stem Cell Regen Biol. 1(1).

Turan, S. et al., 2011. Recombinase-mediated cassette exchange (RMCE): traditional concepts and current challenges. J. Mol. Biol. 407 (2): 193-221.

Any patents or publications mentioned in this specification are indicative of the levels of those skilled in the art to which the invention pertains. One skilled in the art will readily appreciate that the present invention is well adapted to carry out the objects and obtain the ends and advantages mentioned, as well as those inherent therein. The present examples alone with the methods, procedures, molecules, and specific compounds described herein are presently representative of preferred embodiments, are exemplary, and are not limitations on the scope of the invention. Changes therein and other uses will occur to those skill in the art which are encompassed within the spirit of the invention as defined by the scope of the claims.

What is claimed is:

1. A nucleic acid 2A-transcription amplifier, comprising:
a). a first nucleic acid sequence encoding a specific transcription factor (TF) and a gene of interest (GOI), wherein the TF and GOI are operably linked to each other with a nucleic acid sequence encoding a viral 2A-like peptide, wherein the said viral 2A-like peptide separates the said TF and GOI protein during protein translation by the mechanism of ribosomal skidding;
b). a second nucleic acid sequence operably linked to the upstream of the first nucleic acid, wherein the second nucleic acid sequence comprises an upstream activation sequence (UAS) and one promoter;
c). The said promoter regulates the initial expression of the GOI and TF protein, wherein the expressed TF protein specifically binds the UAS region and promotes more TF and GOI protein expression.

2. The 2A-transcription amplifier of claim 1, wherein the transcription factor (TF) is either a natural existing or a synthetic modular protein comprising a DNA-binding domain (DBD) and a trans-activating domain (AD).

3. The 2A-transcription amplifier of claim 1, wherein the gene of interest (GOI) is exogenous or endogenous gene of a eukaryotic cell or organism.
